(19) United States
(12) Patent Application Publication
(10) Pub. No.: US 2015/0119712 A1
(43) Pub. Date: Apr. 30, 2015

(54) ULTRASONIC DIAGNOSTIC DEVICE AND CONTROL PROGRAM THEREFOR

(71) Applicant: GE MEDICAL SYSTEMS GLOBAL TECHNOLOGY COMPANY, LLC, Waukesha, WI (US)

(72) Inventor: Shunichiro Tanigawa, Tokyo (JP)

(73) Assignee: GE MEDICAL SYSTEMS GLOBAL TECHNOLOGY COMPANY, LLC, Waukesha, WI (US)

(21) Appl. No.: 14/522,718

(22) Filed: Oct. 24, 2014

(30) Foreign Application Priority Data
Oct. 29, 2013 (JP) 2013224247

(51) Int. Cl. A61B 8/08; A61B 8/00

(52) U.S. Cl. CPC A61B 8/485; A61B 8/54; A61B 8/4444; A61B 8/461; A61B 8/5223
USPC 600/438

(57) ABSTRACT

An ultrasonic diagnostic device is provided. The ultrasonic diagnostic device includes a transmission control unit configured to control an ultrasonic probe such that transmission of a push pulse of an ultrasonic wave to a biological tissue of a test object and transmission of an ultrasonic pulse for measurement for measuring a shear wave generated in the biological tissue with the push pulse are alternately performed a plurality of times, the transmission control unit configured to control the ultrasonic probe such that an ultrasonic pulse for detection is transmitted, wherein the ultrasonic pulse for detection is for detecting that a first shear wave generated with a first push pulse has passed through a region through which an ultrasonic pulse for measurement corresponding to a second push pulse to be transmitted next to the first push pulse is scheduled to be transmitted.

### CROSS REFERENCE TO RELATED APPLICATIONS

This application claims the benefit of Japanese Patent Application No. 2013-224247 filed Oct. 29, 2013, which is hereby incorporated by reference in its entirety.

### BACKGROUND

The present invention relates to an ultrasonic diagnostic device that transmits a push pulse of an ultrasonic wave so as to measure the elasticity of a biological tissue and a control program therefor.

An elasticity measuring technique of transmitting an ultrasonic pulse (a push pulse) that is high in sound pressure from an ultrasonic probe to a biological tissue so as to measure the elasticity of the biological tissue is known (see, for example, Japanese Patent Application Laid-Open No. 2012-100997). More specifically, a shear wave generated in the biological tissue with the push pulse is detected with an ultrasonic pulse for measurement and a propagation velocity of the shear wave and an elasticity value of the biological tissue are measured. Then, an elastic image having a color according to a calculated value is displayed.

Here, in a case where a two-dimensional elastic image is to be displayed, transmission/reception of ultrasonic pulses for detection for a plurality of sound rays is performed in a two-dimensional measurement region that an elastic image is to be displayed. However, in some cases, it is difficult to measure the shear waves along all the sound rays in the two-dimensional measurement region by single-time push pulse transmission. Therefore, the push pulse is transmitted a plurality of times in order to obtain the elastic images for one frame. Then, the two-dimensional measurement region is segmented into plural ones and in each of the segmented regions, the shear wave generated by the single-time push pulse transmission is detected. The plurality of the push pulses are transmitted at set intervals.

Incidentally, when a transmission interval of the push pulses is too short, there is the possibility that the influence by the previous push pulse transmission may remain to make it difficult to perform accurate measurement. On the other hand, when the transmission interval of the push pulses is too long, there is the possibility that a positional relation between the ultrasonic probe and the biological tissue may be changed between the previous push pulse transmission and the next push pulse transmission to make it difficult to obtain the accurate elastic image. In addition, when the transmission interval of the push pulses is too long, a frame rate is worsened. Thus, it is desirable that the transmission interval of the push pulses be made short to such an extent that the influence by the previous push pulse transmission does not remain. However, since the propagation velocity of the shear wave is varied depending on the elasticity of the biological tissue, it is difficult to set in advance such a transmission interval.

### BRIEF DESCRIPTION

In one aspect, an ultrasonic diagnostic device is provided. The ultrasonic diagnostic device is characterized by including a transmission control unit that controls an ultrasonic probe such that transmission of a push pulse of an ultrasonic wave to a biological tissue of a test object and transmission of an ultrasonic pulse for measurement for measuring a shear wave generated in the aforementioned biological tissue with the push pulse are alternately performed a plurality of times, the transmission control unit that controls the aforementioned ultrasonic probe such that an ultrasonic pulse for detection for detecting that the aforementioned shear wave generated with the aforementioned one push pulse has passed through a region that an ultrasonic pulse for measurement corresponding to another push pulse to be transmitted next to one push pulse is scheduled to be transmitted is transmitted.

According to the above aspect, it is detected with the aforementioned ultrasonic pulse for detection that the aforementioned shear wave generated with the aforementioned one push pulse has passed through the region that the ultrasonic pulse for measurement corresponding to another push pulse to be transmitted next to one push pulse is scheduled to be transmitted. Accordingly, since the next push pulse can be transmitted after the shear wave has passed through the aforementioned region, the next push pulse can be transmitted without being influenced by the previous push pulse transmission.

### BRIEF DESCRIPTION OF THE DRAWINGS

FIG. 1 is a block diagram showing a schematic configuration of an exemplary ultrasonic diagnostic device.

FIG. 2 is a block diagram showing a configuration of an echo data processing unit.

FIG. 3 is a block diagram showing a configuration of a display control unit.

FIG. 4 is a diagram showing a display unit that a B-mode image and an elastic image have been displayed.

FIG. 5 is a diagram showing the display unit that a region of interest has been set on the B-mode image.

FIG. 6 is a diagram showing a transmission sequence of ultrasonic pulses in a case where the elastic image is displayed.

FIG. 7 is a diagram explaining transmission of the push pulse and a shear wave generated with the push pulse.

FIG. 8 is a diagram explaining transmission/reception of an ultrasonic pulse for measurement corresponding to a first-time push pulse transmission.

FIG. 9 is a diagram explaining the order of transmission/reception of the ultrasonic pulses for measurement along a plurality of sound rays.

FIG. 10 is a diagram explaining transmission/reception of an ultrasonic pulse for detection.

FIG. 11 is a diagram explaining transmission/reception of the ultrasonic pulse for measurement corresponding to a second-time push pulse transmission.

FIG. 12 is a diagram showing segmentation of the region of interest in a modified example of a first embodiment.

FIG. 13 is a diagram explaining transmission/reception of the ultrasonic pulse for measurement in a first region.

FIG. 14 is a diagram explaining transmission/reception of the ultrasonic pulse for detection for detecting the shear wave that has passed through a second region.

FIG. 15 is a diagram explaining transmission/reception of the ultrasonic pulse for measurement in the second region.

FIG. 16 is a diagram explaining transmission/reception of the ultrasonic pulse for detection for detecting the shear wave that has passed through a third region.

FIG. 17 is a diagram explaining transmission/reception of the ultrasonic pulse for measurement in the third region.

FIG. 18 is a diagram explaining the first-time push pulse transmission and the shear wave generated with the push pulse in a second embodiment.

FIG. 19 is a diagram explaining transmission/reception of the ultrasonic pulse for measurement corresponding to the first-time push pulse transmission in the second embodiment.

FIG. 20 is a diagram explaining transmission/reception of the ultrasonic pulse for detection in the second embodiment.

FIG. 21 is a diagram explaining the second-time push pulse transmission and the shear wave generated with the push pulse in the second embodiment.

FIG. 22 is a diagram explaining transmission/reception of the ultrasonic pulse for measurement corresponding to the second-time push pulse transmission.

FIG. 23 is a diagram showing another example of a method of transmitting/receiving the ultrasonic pulse for measurement and the ultrasonic pulse for detection.

FIG. 24 is a diagram explaining another example of the position of the ultrasonic pulse for detection.

FIG. 25 is a diagram explaining another example of the position of the ultrasonic pulse for detection.

### DETAILED DESCRIPTION

In the following, exemplary embodiments will be described.

### First Embodiment

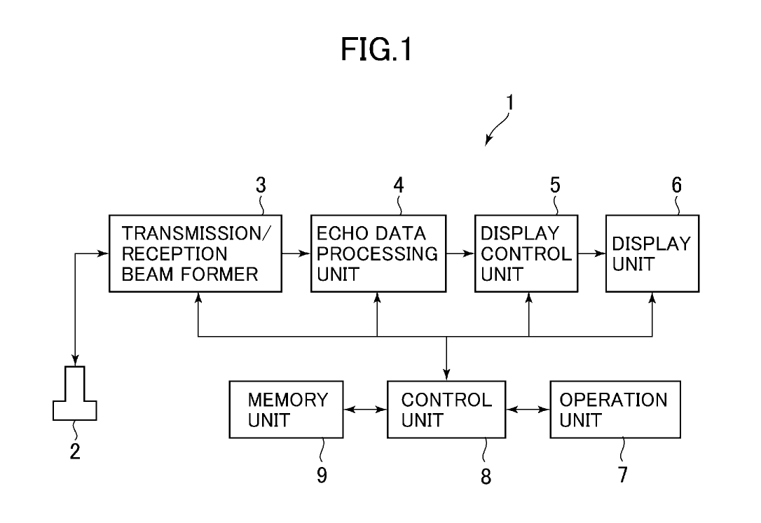

First, a first embodiment will be described. An ultrasonic diagnostic device 1 shown in FIG. 1 is provided with an ultrasonic probe 2, a transmission/reception beam former 3, an echo data processing unit 4, a display control unit 5, a display unit 6, an operation unit 7, a control unit 8 and a memory unit 9.

The aforementioned ultrasonic probe 2 is one example of an embodiment of an ultrasonic probe and transmits an ultrasonic wave to a biological tissue of a test object. An ultrasonic pulse (a push pulse) for generating a shear wave in the biological tissue is transmitted by this ultrasonic probe 2. In addition, an ultrasonic pulse for measurement for measuring the shear wave is transmitted and an echo signal thereof is received by the aforementioned ultrasonic probe 2. As described later, the aforementioned push pulse and the aforementioned ultrasonic pulse for measurement are alternately transmitted a plurality of times.

In addition, as described later, an ultrasonic pulse for detection for detecting the aforementioned shear wave that has passed through a region that the aforementioned push pulse is transmitted next and measurement of the aforementioned shear wave is performed is transmitted by the aforementioned ultrasonic probe 2.

In addition, an ultrasonic pulse for image for creating a B-mode image is transmitted and an echo signal thereof is received by the aforementioned ultrasonic probe 2.

The aforementioned transmission/reception beam former 3 drives the aforementioned ultrasonic probe 2 on the basis of a control signal from the aforementioned control unit 8 to make it transmit the aforementioned various ultrasonic pulses having predetermined transmission parameters (parameter). In addition, the transmission/reception beam former 3 performs signal processing such as phasing addition processing and so forth on the echo signal of the ultrasonic wave.

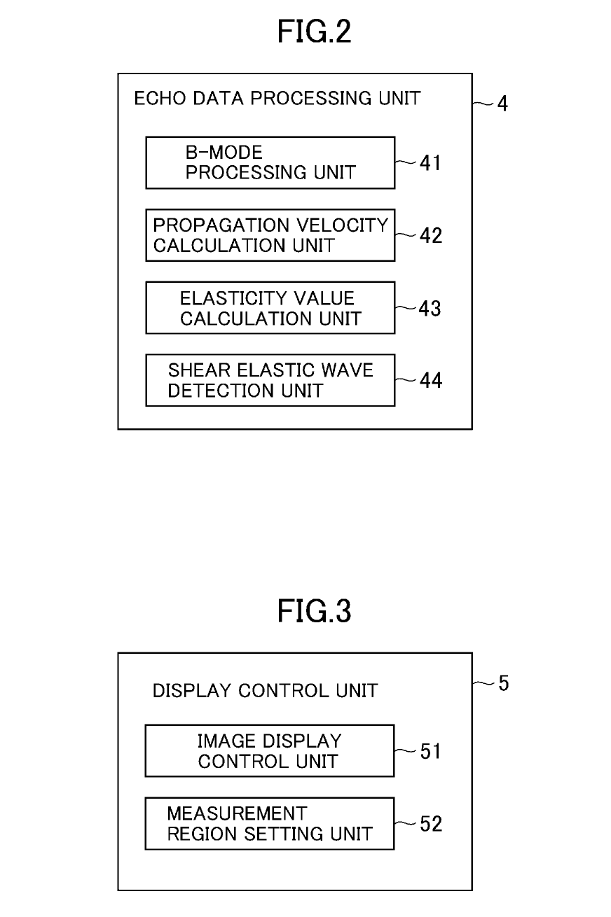

The aforementioned echo data processing unit 4 has a B-mode processing unit 41, a propagation velocity calculation unit 42, an elasticity value calculation unit and a shear wave detection unit 44 as shown in FIG. 2. The aforementioned B-mode processing unit 41 performs B-mode processing such as logarithmic compression processing, envelope detection processing and so forth on echo data output from the aforementioned transmission/reception beam former 3 to create B-mode data.

In addition, the aforementioned propagation velocity calculation unit 42 calculates a propagation velocity of the aforementioned shear wave on the basis of the echo data output from the aforementioned transmission/reception beam former 3. In addition, the aforementioned elasticity value calculation unit 43 calculates the elasticity value of the biological tissue to which the push pulse has been transmitted on the basis of the aforementioned propagation velocity. Details will be described later. The aforementioned propagation velocity calculation unit 42 is one example of an embodiment of a propagation velocity calculation unit. In addition, the aforementioned elasticity value calculation unit 43 is one example of an embodiment of an elasticity value calculation unit. In addition, the aforementioned propagation velocity and the aforementioned elasticity value are examples of embodiments of measured values pertaining to the elasticity of the biological tissue.

Incidentally, only the aforementioned propagation velocity may be calculated and the aforementioned elasticity value may not necessarily be calculated. Data on the aforementioned propagation velocity and data on the aforementioned elasticity value will be referred to as elasticity data.

The aforementioned shear wave detection unit 44 detects the aforementioned shear wave on the basis of the echo signal of the aforementioned ultrasonic pulse for detection. The aforementioned shear wave detection unit 44 is one example of an embodiment of a shear wave detection unit.

The aforementioned display control unit 5 has an image display control unit 51 and a measurement region setting unit 52 as shown in FIG. 3. The aforementioned image display control unit 51 scan-converts the aforementioned B-mode data by a scan converter to create B-mode image data and makes a B-mode image based on this B-mode image data display on the aforementioned display unit 6. In addition, the aforementioned image display control unit 51 scan-converts the aforementioned elasticity data by the scan converter to create elastic image data and makes an elastic image based on this elastic image data display on the aforementioned display unit 6.

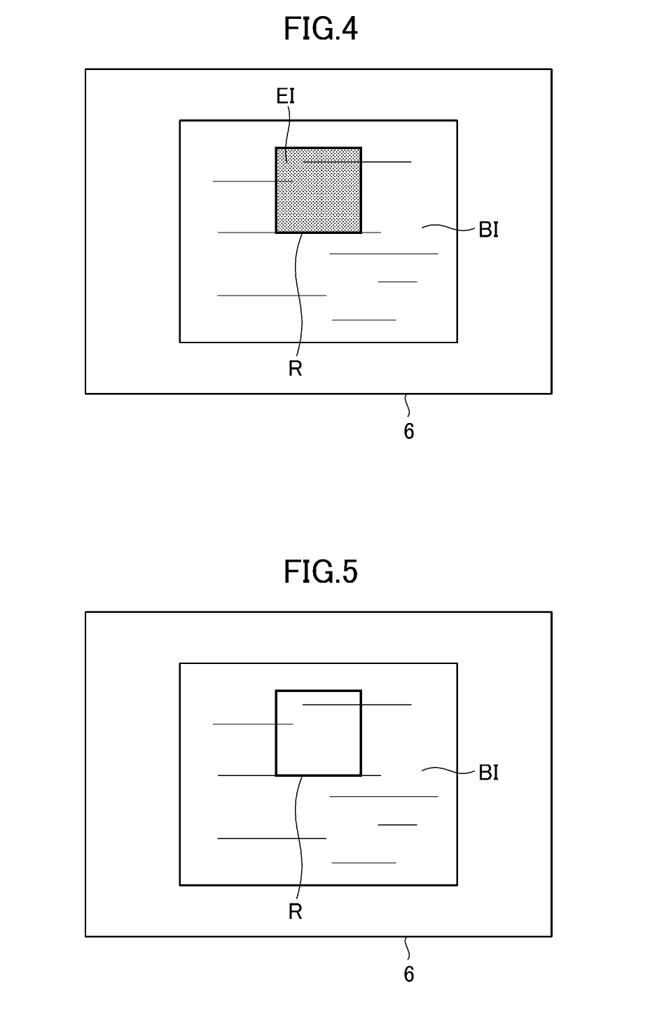

As shown in FIG. 4, the aforementioned elastic image EI is a two-dimensional image to be displayed in a region of interest R set on the aforementioned B-mode image BI. The aforementioned elastic image EI is a color image having a color according to the aforementioned propagation velocity or the aforementioned elasticity value. The aforementioned image display control unit 51 synthesizes together the aforementioned B-mode image data and the aforementioned elastic image data to create synthetic image data and makes an image based on this synthetic image data display on the aforementioned display unit 6. Accordingly, the aforementioned elastic image EI is a semi-transparent image through which the B-mode image BI of the background permeates.

The aforementioned region of interest R is set by the aforementioned measurement region setting unit 52. More specifically, the aforementioned measurement region setting unit 52 sets the aforementioned region of interest R on the basis of an input in the aforementioned operation unit 7 by an operator. The aforementioned region of interest R is a shear wave measurement region and the aforementioned ultrasonic pulse for measurement is transmitted to this region.

The aforementioned display unit 6 is an LCD (Liquid Crystal Display), an organic EL (Electro-Luminescence) display and so forth. Though not shown in the drawing in particular, the aforementioned operation unit 7 is configured by including a keyboard, a pointing device such as a trackball and so forth and others that the operator uses for inputting instructions and information.

Though not shown in the drawing in particular, the aforementioned control unit 8 is configured by having a CPU (Central Processing Unit). This control unit 8 reads out a control program stored in the aforementioned memory unit 9 and makes it execute a function of each unit of the aforementioned ultrasonic diagnostic device 1. For example, the aforementioned control unit 8 outputs a control signal for controlling transmission of the ultrasonic pulse to the aforementioned transmission/reception beam former 3. The aforementioned control unit 8 and the aforementioned transmission/reception beam former 3 are one example of an embodiment of a transmission control unit. In addition, the aforementioned control unit 8 and the aforementioned transmission/reception beam former 3 execute a function that is one example of an embodiment of a transmission control function.

The aforementioned memory unit 9 is an HDD (Hard Disk Drive) or a semiconductor memory (Memory) such as, a RAM (Random Access Memory), a ROM (Read Only Memory) and so forth.

Next, the operation of the ultrasonic diagnostic device 1 of the present example will be described. First, the operator performs transmission/reception of the ultrasonic wave by the aforementioned ultrasonic probe 2 on the test object and makes the B-mode image BI based on the echo signal display as shown in FIG. 5. Then, the region of interest R is set on this B-mode image BI. This region of interest R is set on a region that the elastic image is intended to be displayed.

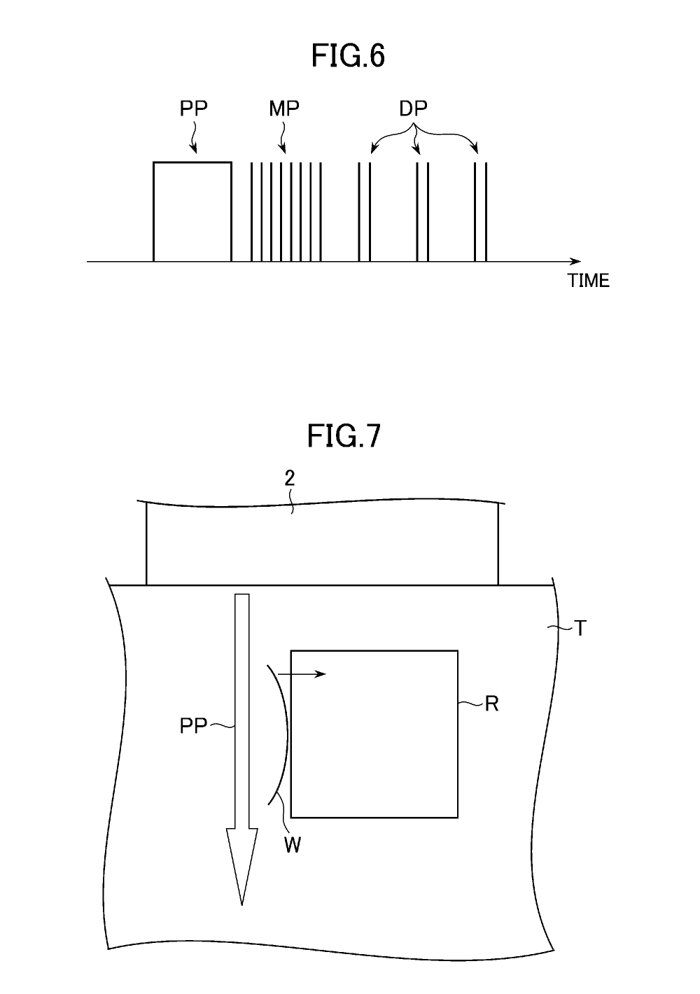

Next, the operator performs input for making the elastic image display by the aforementioned operation unit 7. When this input is made, the aforementioned control unit 8 outputs the control signal to the aforementioned transmission/reception beam former 3 such that ultrasonic pulses are transmitted in order of a push pulse PP, an ultrasonic pulse for measurement MP and an ultrasonic pulse for detection DP as shown in FIG. 6. The ultrasonic pulse for measurement MP and the ultrasonic pulse for detection DP are transmitted a plurality of times for every single-time transmission of the push pulse PP. However, it is not limited to the number of transmissions shown in FIG. 6.

The aforementioned push pulse PP is transmitted a plurality of times (two times in the present example as described later) and the aforementioned ultrasonic pulse for measurement MP and the aforementioned ultrasonic pulse for detection DP are transmitted for every transmission of the push pulse PP. Incidentally in FIG. 6, only the single-time transmission of the push pulse PP is shown.

Transmission of the aforementioned push pulse PP, the aforementioned ultrasonic pulse for measurement MP and the aforementioned ultrasonic pulse for detection DP will be described in detail. Incidentally, in the following explanatory diagrams, the aforementioned push pulse PP, the aforementioned ultrasonic pulse for measurement MP and the aforementioned ultrasonic pulse for detection DP are shown by sound rays (arrows).

The aforementioned push pulse PP is transmitted to the vicinity of the aforementioned region of interest R as shown in FIG. 7. A shear wave W is generated in a biological tissue T with this push pulse PP. This shear wave W propagates in the biological tissue T in a direction (an arrow direction in FIG. 7) going away from the aforementioned push pulse PP and passes through within the aforementioned region of interest R. The shear wave W that propagates in the aforementioned region of interest R is detected with the aforementioned ultrasonic pulse for measurement MP.

Transmission/reception of the aforementioned ultrasonic pulse for measurement MP will be described. In transmission/reception of the aforementioned ultrasonic pulse for measurement MP, the aforementioned region of interest R is segmented into a plurality of regions for convenience sake. Then, in one of the plurality of segmented regions, transmission/reception of the aforementioned ultrasonic pulse for measurement MP is performed by the number of the plurality of sound rays for every single-time transmission of the push pulse PP. Detection of the aforementioned shear wave W is performed in each of the sound rays.

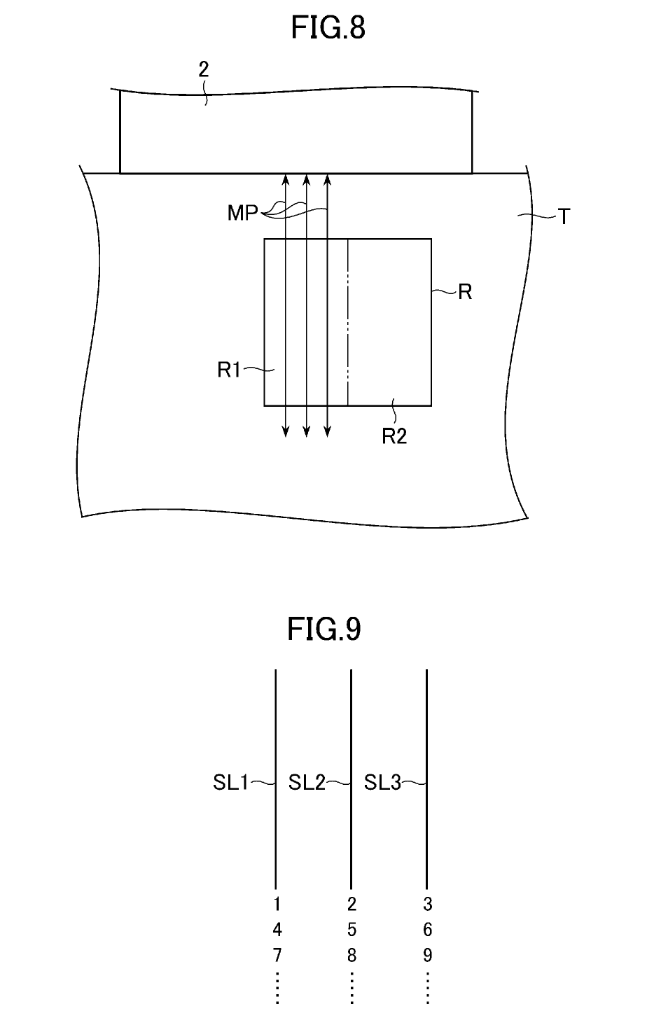

In the present example, as shown in FIG. 8, transmission/reception of the aforementioned ultrasonic pulse for measurement MP is performed with respect to a first region R1 that is one part of the aforementioned measurement region R in a first-time transmission of the aforementioned push pulse PP. The aforementioned first region R1 has a width that is half the width of the aforementioned region of interest R in a propagation direction of the aforementioned shear wave W. The aforementioned first region R1 and the later described aforementioned second region R2 are examples of an embodiment of segmented regions.

In FIG. 8, the ultrasonic pulses for measurement MP for three sound rays are shown. The ultrasonic pulse for measurement MP is transmitted/received along each sound ray a plurality of times. The respective sound rays will be referred to as a first sound ray SL1, a second sound ray SL2 and a third sound ray SL3 as shown in FIG. 9. The aforementioned first sound ray SL1 is the closest to the aforementioned push pulse PP and the aforementioned third sound ray SL3 is the farthest from the aforementioned push pulse PP. Transmission/reception of the aforementioned ultrasonic pulse for measurement MP is performed by an interleaved scanning system in order starting from the one closest to the push pulse PP along each of the sound rays SL1 to SL3. That is, after the ultrasonic pulse for measurement MP has been transmitted/received once at a time in order of the first sound ray SL1, the second sound ray SL2 and the third sound ray SL3, the ultrasonic pulse for measurement MP is again transmitted/received, returning again to the first sound ray SL1. Numerals in FIG. 9 indicate orders that the aforementioned ultrasonic pulses for measurement MP are transmitted/received.

Transmission/reception of the aforementioned ultrasonic pulse for measurement MP is performed a set number of times. This number of times is set to the number of times that the aforementioned shear wave W can be detected in the sound ray that is the farthest from the aforementioned push pulse PP.

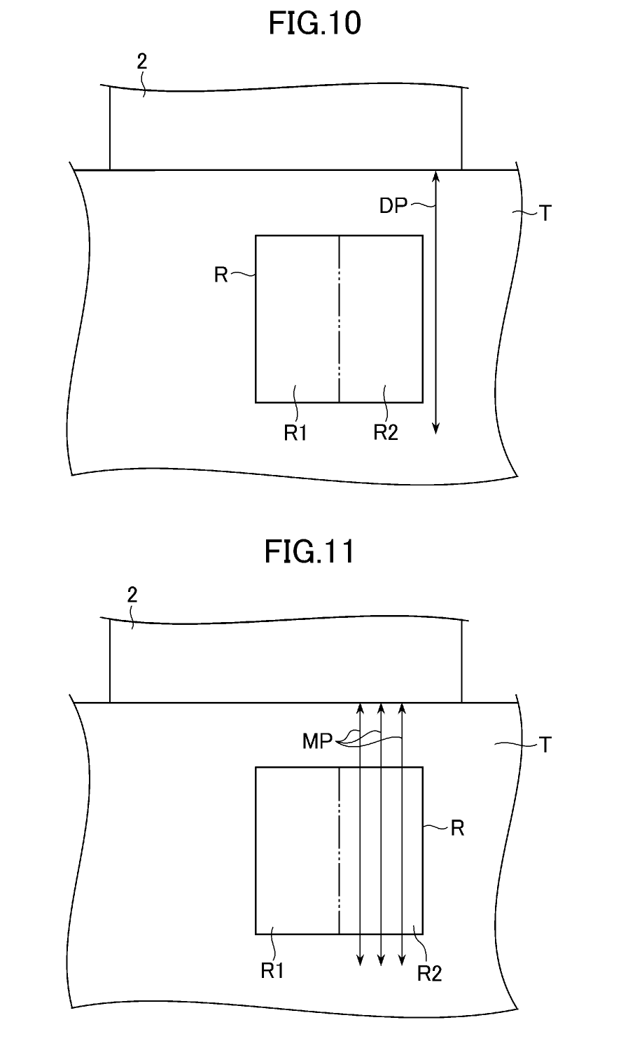

When transmission/reception of the aforementioned ultrasonic pulse for measurement MP is performed the set number of times, transmission/reception of the ultrasonic pulse for detection DP is performed as shown in FIG. 10. The ultrasonic pulse for detection DP is an ultrasonic pulse for detecting the shear wave W that has passed through the second region R2 that the ultrasonic pulse for measurement MP corresponding to the second-time push pulse PP (another push pulse) to be transmitted next to the first-time push pulse PP (one push pulse) is scheduled to be transmitted/received. The shear wave W to be detected here is a shear wave generated by the first-time transmission of the push pulse PP.

The aforementioned ultrasonic pulse for detection DP is transmitted/received in the vicinity of an end located in the propagation direction of the shear wave W in the aforementioned second region R2, the end located farther away from the aforementioned push pulse PP. In the present example, the aforementioned ultrasonic pulse for detection DP is transmitted/received at a position outside the aforementioned second region R2. The transmission/reception position of the aforementioned ultrasonic pulse for detection DP is set in advance.

The aforementioned ultrasonic pulse for detection DP may be transmitted/received the plurality of times. Then, when the shear wave W is detected by the aforementioned shear wave detection unit 44 on the basis of the echo signal of the aforementioned ultrasonic pulse for detection DP, the aforementioned control unit 8 outputs a control signal such that the push pulse PP is again transmitted. Thereby, the second-time push pulse PP is transmitted to the same position as that of the first-time one.

For example, in a case where the shear wave W has been detected on any part of the echo signal of the aforementioned ultrasonic pulse for detection DP for one sound ray, the aforementioned control unit 8 may output the control signal such that the second-time push pulse PP is transmitted.

After the aforementioned second-time push pulse PP has been transmitted, transmission/reception of the ultrasonic wave for measurement MP is performed in the second region R2 as shown in FIG. 11. Transmission/reception of the ultrasonic pulse for measurement MP corresponding to the second-time transmission of the aforementioned push pulse PP is performed in the aforementioned second region R2. Also this transmission/reception of the aforementioned ultrasonic pulse for measurement MP in the second region R2 is performed by the interleaved scanning system along the plurality of sound rays in the same way as in the first region R1.

When the echo signal is obtained by transmission/reception of the ultrasonic pulses for measurement MP in the aforementioned first region R1 and the aforementioned second region R2, the aforementioned propagation velocity calculation unit 42 calculates the propagation velocity of the shear wave W to be detected on the aforementioned echo signal. Incidentally, the aforementioned shear wave W is detected with respect to a part corresponding to a pixel in each of the aforementioned sound rays.

The aforementioned elasticity value calculation unit 43 calculates the elasticity value (a Young's modulus (Pa: Pascal)) on the basis of the aforementioned propagation velocity. However, only the propagation velocity may be calculated with no calculation of the elasticity value.

The aforementioned image display control unit 51 makes the aforementioned elastic image EI display in the aforementioned region of interest R on the aforementioned display unit 6 on the basis of data on the aforementioned propagation velocity or data on the aforementioned elasticity value.

According to the ultrasonic diagnostic device 1 of the present example, after the shear wave W generated with the first-time push pulse PP has been detected, the second-time push pulse PP is transmitted. Therefore, since the second-time push pulse PP is transmitted after the shear wave W generated with the first-time push pulse PP has passed through the aforementioned second region R2, detection of the shear wave W generated with the second-time push pulse PP in the aforementioned second region R2 can be performed without being influenced by the shear wave W generated with the first-time push pulse PP.

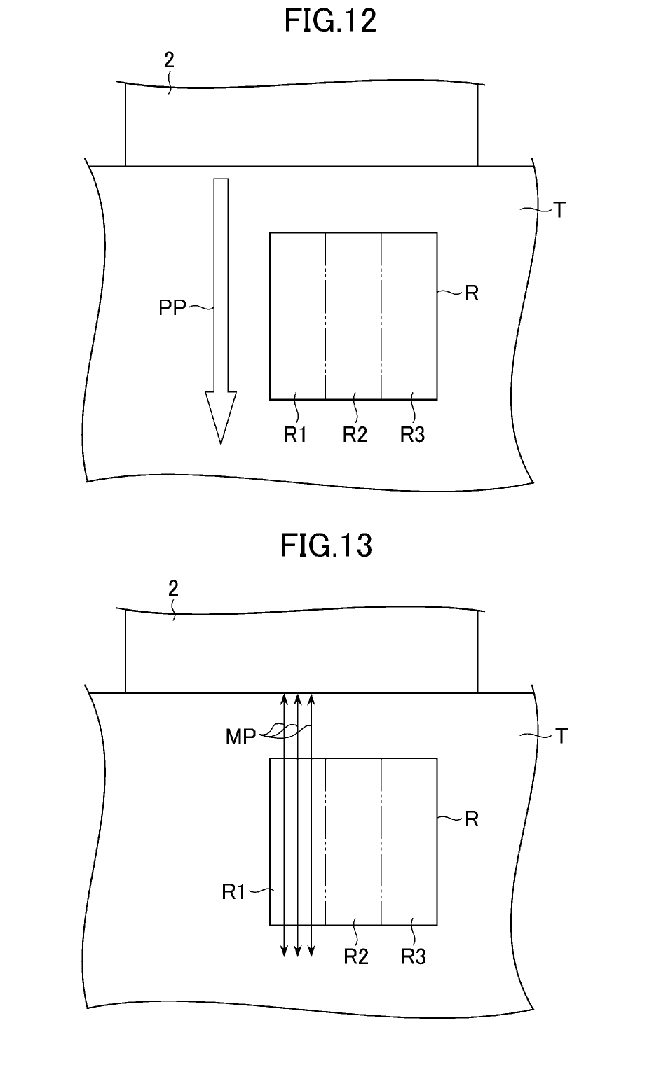

Next, a modified example of the first embodiment will be described. Although in the above-mentioned embodiment, the aforementioned region of interest R is segmented into two of the aforementioned first region R1 and the aforementioned second region R2 and detection of the aforementioned shear wave W is performed by being divided into two, it is not limited to this. For example, as shown in FIG. 12, the aforementioned region of interest R may be segmented into three of the first region R1, the second region R2 and a third region R3 and detection of the aforementioned shear wave W may be detected by being divided into three. In this case, the push pulse PP is transmitted three times and detection of the aforementioned shear wave W is performed in order of the aforementioned first region R1, the aforementioned second region R2 and the aforementioned third region R3.

When transmission/reception of the aforementioned ultrasonic pulse for measurement MP is performed with respect to the aforementioned first region R1 the set number of times as shown in FIG. 13 after the first-time transmission of the push pulse PP, transmission/reception of the ultrasonic pulse for detection DP is performed as shown in FIG. 14. This ultrasonic pulse for detection DP is an ultrasonic pulse for detecting that the shear wave W generated with the first-time push pulse PP has passed through the second region R2 that the ultrasonic pulse for measurement MP corresponding to the second-time push pulse PP to be transmitted next to the firs-time push pulse PP is scheduled to be transmitted/received. The aforementioned ultrasonic pulse for detection DP is transmitted/received in the vicinity of the end located in the propagation direction of the shear wave W in the aforementioned second region R2, the end located farther away from the aforementioned push pulse PP. In the present example, the aforementioned ultrasonic pulse for detection DP is transmitted/received outside the aforementioned second region R2 and in the aforementioned third region R3. The transmission/reception position of the aforementioned ultrasonic pulse for detection DP is set in advance.

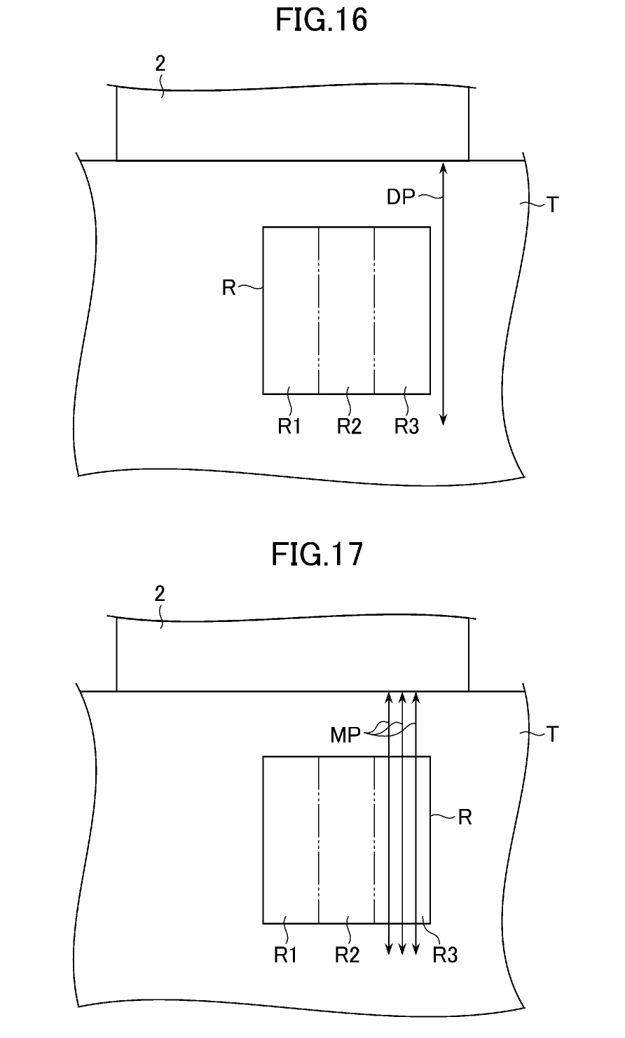

However, the aforementioned ultrasonic pulse for detection DP may be transmitted/received at a position where the shear wave W that has passed through the aforementioned second region R2 can be detected and may be transmitted/received, for example, outside (the same position as that in the later described FIG. 16) the aforementioned third region R3.

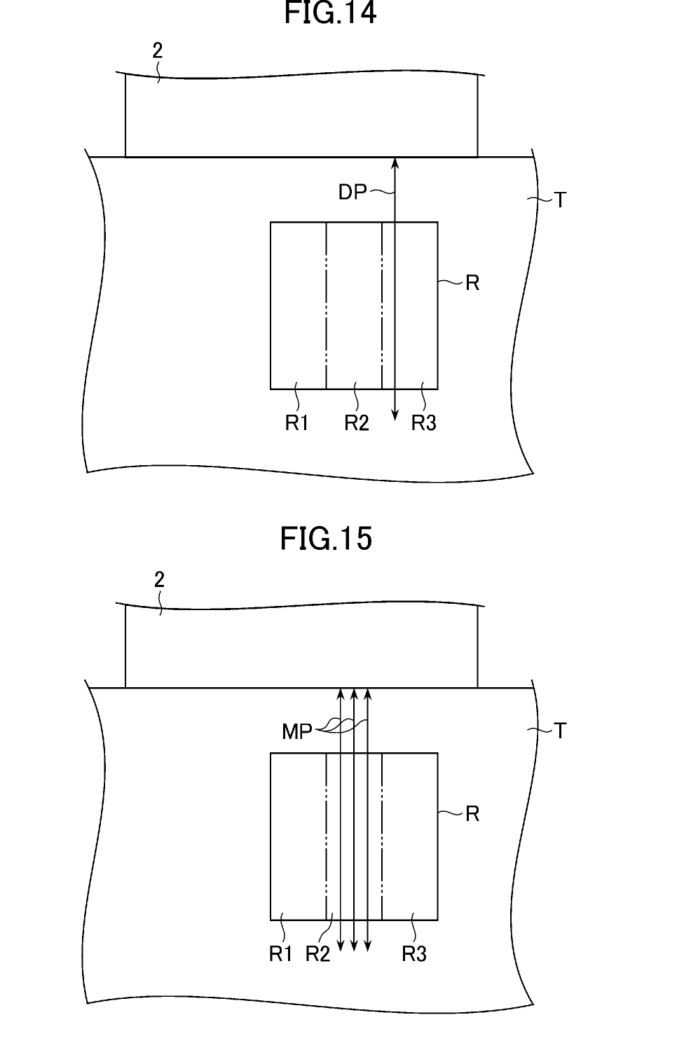

When the shear wave W generated by the first-time transmission of the push pulse PP is detected with the aforementioned ultrasonic pulse for detection DP, the push pulse PP is again transmitted in the same way as in the above-mentioned embodiment. Thereafter, transmission/reception of the ultrasonic pulse for measurement MP is performed in the aforementioned second region R2 as shown in FIG. 15. When this transmission/reception of the ultrasonic pulse for measurement MP is performed the set number of times, transmission/reception of the ultrasonic pulse for detection DP is performed as shown in FIG. 16. This ultrasonic pulse for detection DP is an ultrasonic pulse for detecting that the shear wave W generated with the second-time push pulse PP has passed through the third region R3 that the ultrasonic pulse for measurement MP corresponding to the third-time push pulse PP to be transmitted next to the second-time push pulse PP is scheduled to be transmitted/received. The aforementioned ultrasonic pulse for detection DP is transmitted/received in the vicinity of an end located in the propagation direction of the shear wave W in the aforementioned third region R3, the end located farther away from the aforementioned push pulse PP. In the present example, the aforementioned ultrasonic pulse for detection DP is transmitted/received at the position outside the aforementioned third region R3. The transmission/reception position of the aforementioned ultrasonic pulse for detection DP is set in advance.

When the shear wave W generated by the second-time transmission of the push pulse PP is detected with the aforementioned ultrasonic pulse for detection DP, the push pulse PP is again transmitted and thereafter transmission/reception of the ultrasonic pulse for measurement MP is performed in the aforementioned third region R3 as shown in FIG. 17.

### Second Embodiment

Next, a second embodiment will be described. The configuration of the ultrasonic diagnostic device of the second embodiment is the same as that of the first embodiment and, in the following, matters different from those in the first embodiment in operation will be described.

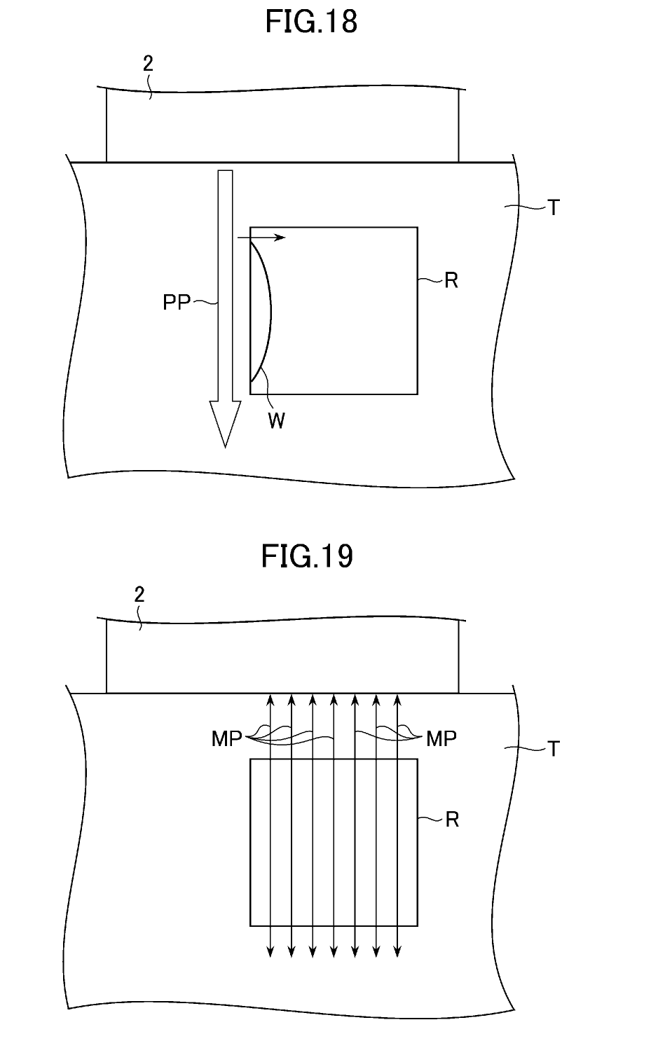

Although transmission/reception of the push pulse PP is performed the plurality of times also in the present example, transmission/reception of the ultrasonic pulse for measurement MP corresponding to transmission of each push pulse is performed over the entire of the aforementioned region of interest R. Description will be made specifically. After the push pulse PP has been transmitted to the vicinity of the aforementioned region of interest R as shown in FIG. 18, transmission/reception of the ultrasonic pulse for measurement MP is performed over the entire of the aforementioned region of interest R as shown in FIG. 19.

Also in the present example, transmission/reception of the aforementioned ultrasonic pulse for measurement MP is performed by the interleaved scanning system in order starting from the one closer to the aforementioned push pulse PP in the same way as in the first embodiment. In addition, transmission/reception of the aforementioned ultrasonic pulse for measurement MP is performed the plurality of times with respect to each of the plurality of sound rays.

However, the aforementioned ultrasonic pulse for measurement MP (a plane wave) may be transmitted once to a region including the entire of the aforementioned region of interest R. In this case, the aforementioned transmission/reception beam former 3 performs plural sound rays parallel processing to acquire the echo signals for the plurality of sound rays over the entire of the aforementioned region of interest R.

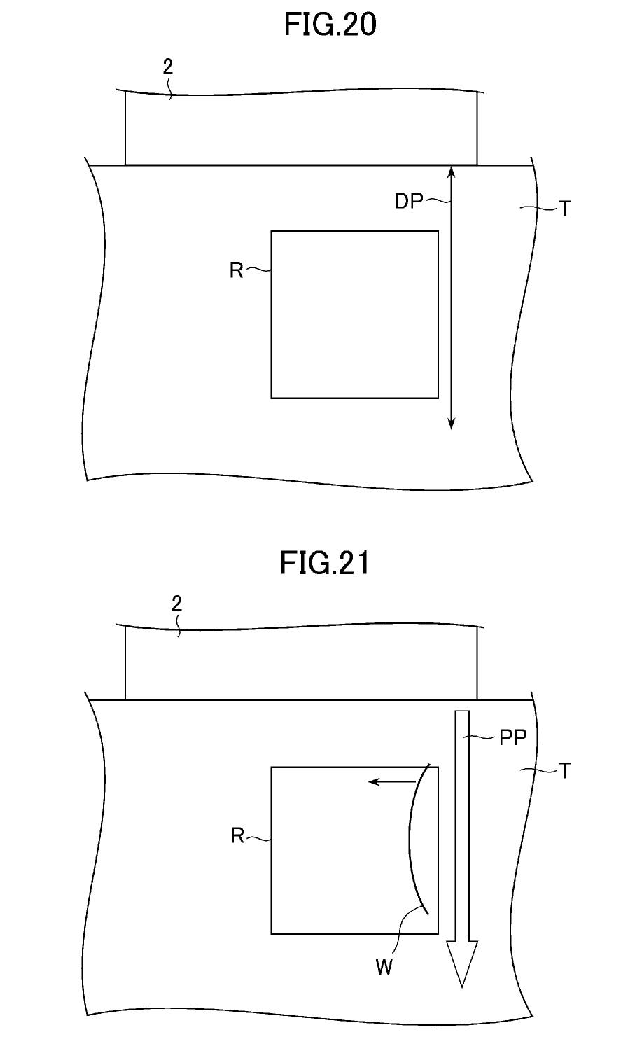

When transmission/reception of the aforementioned ultrasonic pulse for measurement MP is performed the set number of times, transmission/reception of the ultrasonic pulse for detection DP is performed as shown in FIG. 20. This ultrasonic pulse for detection DP is an ultrasonic pulse for detecting the shear wave W that has passed through the aforementioned region of interest R that the ultrasonic pulse for measurement MP corresponding to the second-time push pulse PP to be transmitted next to the first-time push pulse PP is scheduled to be transmitted/received. Also the shear wave W to be detected here is the shear wave generated by the first-time transmission of the push pulse PP as in the first embodiment.

The aforementioned ultrasonic pulse for detection DP is transmitted/received in the vicinity of an end located in the propagation direction of the shear wave W in the aforementioned region of interest R, the end located farther away from the aforementioned first-time push pulse PP. The position of the aforementioned ultrasonic pulse for detection DP is the position that is located outside the region of interest R and is the same as that in the first embodiment.

When the shear wave is detected by the aforementioned shear wave detection unit 44 on the basis of the echo signal of the aforementioned ultrasonic pulse for detection DP, the aforementioned control unit 8 outputs the control signal such that the push pulse PP is again transmitted. In the present example, the second-time push pulse PP is transmitted to the side opposite to the position of the first-time push pulse PP relative to the aforementioned region of interest R as shown in FIG. 21. The propagation direction of the shear wave W in the aforementioned region of interest R is oriented in opposite directions between the first-time and second-time ones because the positions of the first-time and second-time push pulses PP are made different from each other in this way.

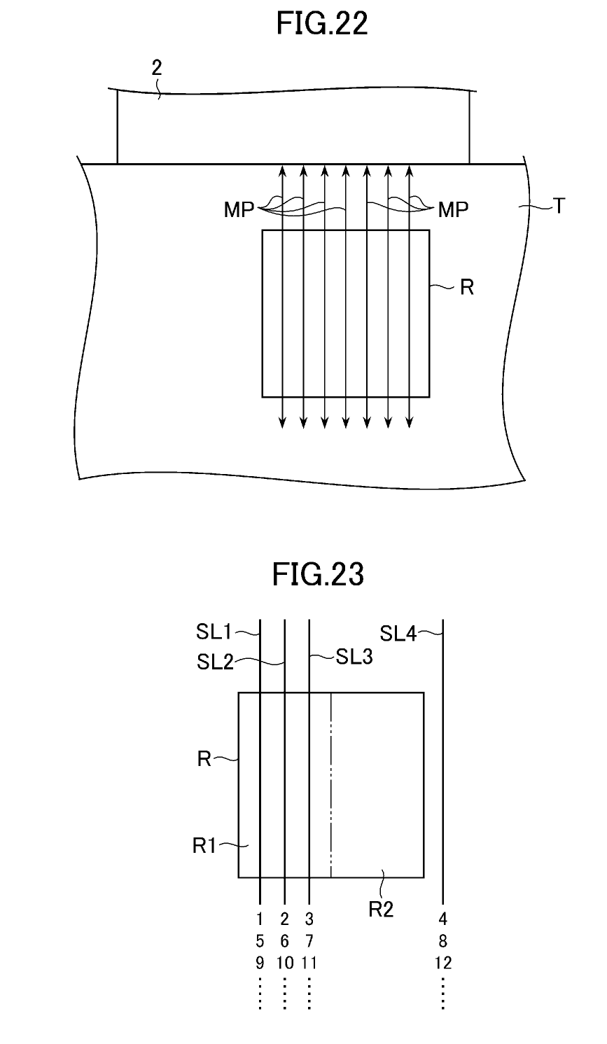

After the aforementioned second-time push pulse PP has been transmitted, transmission/reception of the ultrasonic pulse for measurement MP is performed over the entire of the aforementioned region of interest R as shown in FIG. 22. This transmission/reception of the ultrasonic pulse for measurement MP is performed by the interleaved scanning system in order starting from the side closer to the second-time push pulse PP. Therefore, the order of transmission/reception along the plurality of sound rays is made different between the first-time and second-time ones. In addition, transmission/reception of the aforementioned ultrasonic pulse for measurement MP is performed the plurality of times for each of the plurality of sound rays.

However, similarly to the first-time one, transmission of the aforementioned ultrasonic pulse for measurement MP may be performed only once and the echo signals for the plurality of sound rays may be acquired by the plural sound rays parallel processing.

When the echo signals are acquired by the first-time and second-time transmissions/receptions of the ultrasonic pulses for measurement MP, the aforementioned propagation velocity calculation unit 42 calculates propagation velocities of the shear waves W detected with the aforementioned echo signals. The aforementioned propagation velocity calculation unit 42 calculates a propagation velocity V1 on the basis of the echo signal obtained by the first-time transmission/reception of the ultrasonic pulse for measurement MP. In addition, the aforementioned propagation velocity calculation unit 42 calculates a propagation velocity V2 also on the basis of the echo signal obtained by the second-time transmission/reception of the ultrasonic pulse for measurement MP.

The aforementioned elasticity value calculation unit 43 calculates the elasticity value E1 on the basis of the aforementioned propagation velocity V1. In addition, the aforementioned elasticity value calculation unit 43 calculates an elasticity value E2 on the basis of the aforementioned propagation velocity V2.

In a case where the elastic image EI based on the data on the propagation velocity is to be displayed, the aforementioned image display control unit 51 performs additional averaging processing on data on the aforementioned propagation velocity V1 and data on the aforementioned propagation velocity V2 with respect to a corresponding pixel position. Then, it makes the elastic image EI of one frame display on the aforementioned display unit 6 on the basis of data obtained by this additional averaging processing.

In addition, in a case where the elastic image EI based on data on the elasticity value is to be displayed, the aforementioned image display control unit 51 performs the additional averaging processing on data on the aforementioned elasticity value E1 and data on the aforementioned elasticity value E2 with respect to the corresponding pixel position. Then, it makes the elastic image EI of one frame display on the aforementioned display unit on the basis of data obtained by this additional averaging processing.

According to the present example, similarly to the first embodiment, after the shear wave W generated with the first-time push pulse PP has been detected, the second-time push pulse PP is transmitted. Therefore, since the second-time push pulse PP is transmitted after the shear wave W generated with the first-time push pulse PP has passed through the aforementioned region of interest R, detection of the shear wave W generated with this second-time push pulse PP can be performed without being influenced by the shear wave W generated with the first-time push pulse PP.

Although the disclosure has been described in accordance with the aforementioned exemplary embodiments as mentioned above, the systems and methods described herein can be modified in a variety of ways within a range not changing the gist thereof. For example, although in the above-mentioned embodiments, when transmission/reception of the aforementioned ultrasonic pulse for measurement MP is performed the set number of times, transmission/reception of the aforementioned ultrasonic pulse for detection DP is performed, transmission/reception of the aforementioned ultrasonic pulse for detection DP may be performed when the shear wave is detected with the aforementioned ultrasonic pulse for measurement MP along the sound ray that is the farthest from the aforementioned pushing pulse PP.

In addition, transmission/reception of the ultrasonic pulse for measurement MP and the ultrasonic pulse for detection DP may be performed by the interleaved scanning system. That is, after each single-time transmission/reception of the ultrasonic pulse for measurement MP has been performed for all the sound rays along which transmission/reception of the ultrasonic pulses for measurement MP is performed for every single-time transmission of the push pulse PP, transmission/reception of the ultrasonic pulse for detection DP may be performed. For example, as shown in FIG. 23, the sound rays along which transmission/reception of the ultrasonic pulses for measurement MP is performed in the first region R1 will be referred to as the first sound ray SL1, the second sound ray SL2 and the third sound ray SL3, and a sound ray along which transmission/reception of the ultrasonic pulse for detection DP is performed will be referred to as a fourth sound ray SL4. They are closer to the aforementioned push pulse PP in order of the aforementioned first sound ray SL1, the aforementioned second sound ray SL2, the aforementioned third sound ray SL3 and the aforementioned fourth sound ray SL4.

The order of transmission/reception of the aforementioned ultrasonic pulse for measurement MP and the aforementioned ultrasonic pulse for detection DP will be described. After the ultrasonic pulse for measurement MP has been transmitted/received once at a time in order of the first sound ray SL1, the second sound ray SL2 and the third sound ray SL3, the ultrasonic pulse for detection DP is transmitted/received along the aforementioned fourth sound ray SL4. Thereafter, the ultrasonic pulse for measurement MP is transmitted/received returning again to the first sound ray SL1. The numerals in FIG. 23 indicate the orders that the aforementioned ultrasonic pulses for measurement MP are transmitted/received.

In addition, the position of the aforementioned ultrasonic pulse for detection DP is not limited to the position in the above-mentioned embodiments. The position of the aforementioned ultrasonic pulse for detection DP may be a position where it can be detected that the shear wave generated with the aforementioned one push pulse has passed through the region that the ultrasonic pulse for measurement MP corresponding to another push pulse to be transmitted next to one push pulse is scheduled to be transmitted/received. For example, as shown in FIG. 24, in a case where the region that the ultrasonic pulse for measurement MP corresponding to the push pulse to be transmitted next is to be transmitted/received is the second region R2, the aforementioned ultrasonic pulse for detection DP may be transmitted/received on an end located in the propagation direction of the shear wave W in this second region R2, the end located farther away from the push pulse. The shear wave W that has passed through the aforementioned second region R2 can be detected also by transmitting/receiving the aforementioned ultrasonic pulse for detection DP at such a position.

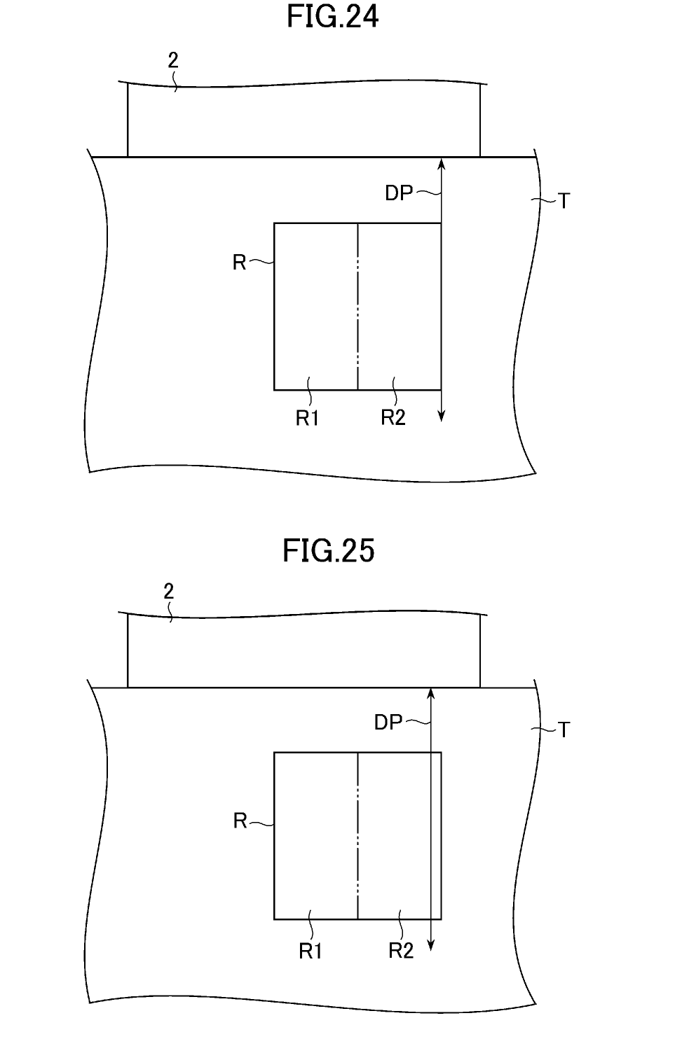

In addition, similarly, in a case where the region that the ultrasonic pulse for measurement MP corresponding to the next push pulse PP is to be transmitted/received is the second region R2, transmission/reception of the aforementioned ultrasonic pulse for detection DP may be performed in this second region R2 as shown in FIG. 25. In this case, the aforementioned ultrasonic pulse for detection DP is transmitted/received in the vicinity of the end located in the propagation direction of the shear wave W in the aforementioned second region R2, the end located farther away from the aforementioned push pulse PP. The shear wave W that has passed through the aforementioned second region R2 can be detected also by transmitting/receiving the aforementioned ultrasonic pulse for detection DP at such a position. Here, not only that the shear wave W has passed through the entire of the aforementioned second region R2 and has gone out of the second region R2 but also that it has nearly passed through the second region R2 are included in “passed”.

1. An ultrasonic diagnostic device comprising a transmission control unit configured to control an ultrasonic probe such that transmission of a push pulse of an ultrasonic wave to a biological tissue of a test object and transmission of an ultrasonic pulse for measurement for measuring a shear wave generated in the biological tissue with the push pulse are alternately performed a plurality of times,
the transmission control unit configured to control the ultrasonic probe such that an ultrasonic pulse for detection is transmitted, wherein the ultrasonic pulse for detection is for detecting that a first shear wave generated with a first push pulse has passed through a region through which an ultrasonic pulse for measurement corresponding to a second push pulse to be transmitted next to the first push pulse is scheduled to be transmitted.

2. The ultrasonic diagnostic device defined in claim 1, comprising:
a shear wave detection unit configured to detect the first shear wave based on an echo signal obtained by transmission of the ultrasonic pulse for detection,
wherein the transmission control unit is configured to control the ultrasonic probe so as to transmit the second push pulse when the first shear wave is detected by the shear wave detection unit.

3. The ultrasonic diagnostic device defined in claim 1,
wherein the region is one of a plurality of segmented regions segmented from a measurement region for which a measured value pertaining to an elasticity of the biological tissue is calculated based on the detection of the first shear wave, and for which an elastic image of the biological tissue is displayed,
wherein the transmission control unit is configured to control the ultrasonic probe such that the ultrasonic pulse for measurement is transmitted in one of the plurality of segmented regions for every single-time transmission of the push pulse, and
wherein the transmission control unit is configured to control the ultrasonic probe such that the ultrasonic pulse for detection is transmitted for detecting that the first shear wave generated with the first push pulse has passed through a segmented region of the plurality of segmented regions through which an ultrasonic pulse for measurement corresponding to the second push pulse is scheduled to be transmitted.

4. The ultrasonic diagnostic device defined in claim 2,
wherein the region is one of a plurality of segmented regions segmented from a measurement region for which a measured value pertaining to an elasticity of the biological tissue is calculated based on the detection of the first shear wave, and for which an elastic image of the biological tissue is displayed,
wherein the transmission control unit is configured to control the ultrasonic probe such that the ultrasonic pulse for measurement is transmitted in one of the plurality of segmented regions for every single-time transmission of the push pulse, and
wherein the transmission control unit is configured to control the ultrasonic probe such that the ultrasonic pulse for detection is transmitted for detecting that the first shear wave generated with the first push pulse has passed through a segmented region of the plurality of segmented regions through which an ultrasonic pulse for measurement corresponding to the second push pulse is scheduled to be transmitted.

5. The ultrasonic diagnostic device defined in claim 3,
wherein the transmission control unit is configured to control the ultrasonic probe such that the ultrasonic pulse for detection is transmitted to a position that has been set in advance in the vicinity of an end located in a propagation direction of the shear elastic wave in the segmented region, the end located farther away from the push pulse.

6. The ultrasonic diagnostic device defined in claim 4,
wherein the transmission control unit is configured to control the ultrasonic probe such that the ultrasonic pulse for detection is transmitted to a position that has been set in advance in the vicinity of an end located in a propagation direction of the shear elastic wave in the segmented region, the end located farther away from the push pulse.

7. The ultrasonic diagnostic device defined in claim 1,
wherein the region is a measurement region for which a value pertaining to an elasticity of the biological tissue is measured based on detection of the shear wave, and for which an elastic image of the biological tissue is displayed,
wherein the transmission control unit is configured to control the ultrasonic probe such that the ultrasonic pulse for measurement is transmitted in the measurement region for every single-time transmission of the push pulse, and
wherein the transmission control unit is configured to control the ultrasonic probe such that the ultrasonic pulse for detection is transmitted for detecting the first shear wave that has passed through the measurement region.

8. The ultrasonic diagnostic device defined in claim 2,
wherein the region is a measurement region for which a value pertaining to an elasticity of the biological tissue is measured based on detection of the shear wave, and for which an elastic image of the biological tissue is displayed,
wherein the transmission control unit is configured to control the ultrasonic probe such that the ultrasonic pulse for measurement is transmitted in the measurement region for every single-time transmission of the push pulse, and
wherein the transmission control unit is configured to control the ultrasonic probe such that the ultrasonic pulse for detection is transmitted for detecting the first shear wave that has passed through the measurement region.

9. The ultrasonic diagnostic device defined in claim 7,
wherein the transmission control unit is configured to control the ultrasonic probe such that the ultrasonic pulse for detection is transmitted to a position that has been set in advance in the vicinity of an end located in a propagation direction of the shear wave in the measurement region, the end located farther away from the push pulse.

10. The ultrasonic diagnostic device defined in claim 8,
wherein the transmission control unit is configured to control the ultrasonic probe such that the ultrasonic pulse for detection is transmitted to a position that has been set in advance in the vicinity of an end located in a propagation direction of the shear wave in the measurement region, the end located farther away from the push pulse.

11. The ultrasonic diagnostic device defined in claim 1, comprising a propagation velocity calculation unit configured to calculate a propagation velocity of the shear wave as a measured value pertaining to an elasticity of the biological tissue based on an echo signal of the ultrasonic pulse for measurement.

12. The ultrasonic diagnostic device defined in claim 2, comprising a propagation velocity calculation unit configured to calculate a propagation velocity of the shear wave as a measured value pertaining to the elasticity of the biological tissue based on an echo signal of the ultrasonic pulse for measurement.

13. The ultrasonic diagnostic device defined in claim 3, comprising a propagation velocity calculation unit configured to calculate a propagation velocity of the shear wave as a measured value pertaining to the elasticity of the biological tissue based on an echo signal of the ultrasonic pulse for measurement.

14. The ultrasonic diagnostic device defined in claim 11, comprising an elasticity value calculation unit configured to calculate an elasticity value of the biological tissue as the measured value pertaining to the elasticity of the biological tissue based on the propagation velocity of the shear wave.

15. The ultrasonic diagnostic device defined in claim 12, comprising an elasticity value calculation unit configured to calculate an elasticity value of the biological tissue as the measured value pertaining to the elasticity of the biological tissue based on of the propagation velocity of the shear wave.

16. The ultrasonic diagnostic device defined in claim 13, comprising an elasticity value calculation unit configured to calculate an elasticity value of the biological tissue as the measured value pertaining to the elasticity of the biological tissue based on the propagation velocity of the shear wave.

17. The ultrasonic diagnostic device defined in claim 14, comprising a display unit configured to display a two-dimensional elastic image having a display form according to the propagation velocity or the elasticity value.

18. The ultrasonic diagnostic device defined in claim 15, comprising a display unit configured to display a two-dimensional elastic image having a display form according to the propagation velocity or the elasticity value.

19. An ultrasonic diagnostic device comprising a central processing unit programmed to:
control an ultrasonic probe such that transmission of a push pulse of an ultrasonic wave to a biological tissue of a test object and transmission of an ultrasonic pulse for measurement for measuring a shear wave generated in the biological tissue with the push pulse are alternately performed a plurality of times,
control the ultrasonic probe such that an ultrasonic pulse for detection is transmitted, wherein the ultrasonic pulse for detection is for detecting that a first shear wave generated with a first push pulse has passed through a region through which an ultrasonic pulse for measurement corresponding to a second push pulse to be transmitted next to the first push pulse is scheduled to be transmitted.

20. A control program for ultrasonic diagnostic device, the control program configured to cause a computer to execute:
a transmission control function of controlling an ultrasonic probe such that transmission of a push pulse of an ultrasonic wave to a biological tissue of a test object and transmission of an ultrasonic pulse for measurement for measuring a shear wave generated in the biological tissue with the push pulse are alternately performed a plurality of times,
the transmission control function of controlling the ultrasonic probe such that an ultrasonic pulse for detection is transmitted, wherein the ultrasonic pulse for detection is for detecting that a first shear wave generated with a first push pulse has passed through a region through which an ultrasonic pulse for measurement corresponding to a second push pulse to be transmitted next to the first push pulse is scheduled to be transmitted.
